(19) United States
(12) Patent Application Publication
(10) Pub. No.: US 2026/0183322 A1
(43) Pub. Date: Jul. 2, 2026

(54) BORON-BASED ENOLASE LIGANDS AND METHODS OF USE FOR TREATMENT OF CANCER

(71) Applicant: LOMA LINDA UNIVERSITY, Loma Linda, CA (US)

(72) Inventors: Carlos CASIANO, Loma Linda, CA (US); Bhaskar DAS, Loma Linda, CA (US); Frankis ALMAGUEL, Loma Linda, CA (US)

(73) Assignee: LOMA LINDA UNIVERSITY, Loma Linda, CA (US)

(21) Appl. No.: 19/128,346

(22) PCT Filed: Nov. 9, 2023
(86) PCT No.: PCT/US2023/079286

§ 371 Date: May 8, 2025

Related U.S. Application Data
(60) Provisional application No. 63/383,015, filed on Nov. 9, 2022.

(51) Int. Cl. A61K 31/69; A61P 35/00

(52) U.S. Cl. CPC A61K 31/69; A61P 35/00

(57) ABSTRACT

Pharmaceutical compositions and methods for using boron-based ligands of enolases to treat cancer are disclosed. One such pharmaceutical composition includes a therapeutically effective amount of a ligand of enolase (1) having a general formula III or a pharmaceutically acceptable derivative thereof. The cancer can be a carcinoma, sarcoma, lymphoma, leukemia, or melanoma. The cancer can be prostate cancer.

### TECHNICAL FIELD

The disclosure relates to pharmaceutical compositions targeting enolase (ENO) and methods of treatment of cancer using these pharmaceutical compositions. More specifically, the disclosure relates to boron-based ligands of ENO.

### BACKGROUND

The use of boron compounds in pharmaceutical drug design has been limited. Due to the boron atom vacant orbital, the boron atom can inter-convert between the neutral sp² and the anionic sp³ hybridization states to generate a stable interaction between the boron atom and donor molecules. As a result, boron atoms interact with target proteins producing potent biological activities after the atom is introduced into biologically active molecular frameworks. Boron atoms have antioxidant properties by scavenging Reactive Oxygen Species (ROS).

ENO has three isoforms: ENO1 (alpha-enolase) that is expressed in most human tissues with high tumor expression, ENO2 (γ-enolase or neuron-specific enolase, NSE) that is expressed in neuroendocrine differentiated PCa (NEPC), and muscle-specific ENO3 (beta-enolase). ENO1 and ENO2 share structural and functional similarity and dimerize to exert their biological functions. ENO1 (47 kD) has a smaller variant (37 kD) called c-MYC binding protein 1 (MPB1), which lacks glycolytic activity and functions as a negative regulator. ENO1 or alpha-enolase is upregulated in cancer cells by the c-MYC oncogene under elevated glucose levels to drive aerobic glycolysis (the Warburg effect). Along with its role in glycolysis, ENO1 has other tumor functions including cell surface-related plasminogen activity contributing to cancer cell migration, invasion, and metastasis, protein interactions in the cell surface to regulate glycolysis, promotion of oncogenic signaling, anti-tumor immune modulation, and chemotherapy resistance. Pre-clinical studies targeting ENO1 with antibody-based immunotherapy in pancreatic cancer have shown moderate effectiveness. But, there remains a need for pharmacological approaches using ligands, such as small molecule inhibitors (SMIs), and other therapeutic agents.

### SUMMARY

Provided here are pharmaceutical compositions and methods to address the shortcomings of the art and provide other additional or alternative advantages. The disclosure provides several embodiments of pharmaceutical compositions containing boron-based ligands and methods of treating cancer in a subject. An embodiment of a pharmaceutical composition includes a therapeutically effective amount of a ligand of enolase 1. The ligand has a general formula I or a pharmaceutically acceptable derivative thereof:

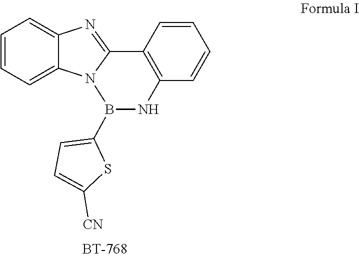

Embodiments of a method of treating cancer in a subject includes administering to the subject the pharmaceutical composition containing a therapeutically effective amount of a ligand of general formula I or a pharmaceutically acceptable derivative thereof. The cancer can be carcinoma, sarcoma, lymphoma, leukemia, or melanoma. The cancer can be of a bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In certain examples, the cancer is prostate cancer.

Another embodiment of a pharmaceutical composition includes a therapeutically effective amount of a ligand of enolase 1 having a general formula II or a pharmaceutically acceptable derivative thereof:

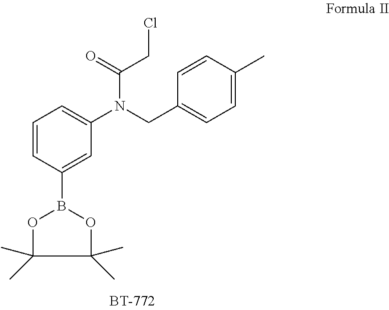

Another embodiment of a method of treating cancer in a subject includes administering to the subject the pharmaceutical composition containing a therapeutically effective amount of a ligand of general formula II or a pharmaceutically acceptable derivative thereof. The cancer can be carcinoma, sarcoma, lymphoma, leukemia, or melanoma. The cancer can be of a bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In certain examples, the cancer is prostate cancer.

Another embodiment of a pharmaceutical composition includes a therapeutically effective amount of a ligand of enolase 1 having a general formula III or a pharmaceutically acceptable derivative thereof:

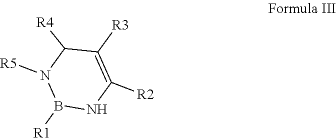

where R1 is an aliphatic moiety, R2 is an aromatic moiety, R3 is a heterocyclic moiety, R4 is a cyano group, and R5 is an acid moiety or a derivative thereof.

Another embodiment of a method of treating cancer in a subject includes administering to the subject the pharmaceutical composition containing a therapeutically effective amount of a ligand of general formula III. The cancer can be carcinoma, sarcoma, lymphoma, leukemia, or melanoma. The cancer can be of a bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In certain examples, the cancer is prostate cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. To facilitate this description, like referenced numerals designate like structural elements or procedures in a method. Embodiments are illustrated by way of example and not by way of limitation in the figures of the accompanying drawings.

FIG. 1 is a schematic representation of the general of structure of 10 compounds synthesized from Diazaborine-based pharmacophore group compounds, in accordance with an embodiment of the disclosure.

FIG. 2A is a schematic representation of the synthesis of diazaborinine, in accordance with an embodiment of the disclosure.

FIG. 2B is a schematic representation of the synthesis of diazaborine, in accordance with various embodiments.

FIG. 3A is a graphical representation of 3-(4,5-dimethylthiazol-2-yl)-2,5-diphenyl-2H-tetrazolium bromide (MTT) assay-based dose response curve for the ligand BT-772 in the PC3 prostate cancer cell line and its taxane resistant variant PC3-DR, in accordance with an embodiment of the disclosure.

FIG. 3B is a graphical representation of 3-(4,5-dimethylthiazol-2-yl)-2,5-diphenyl-2H-tetrazolium bromide MTT assay-based dose response curve for the ligand BT-772 in the DU145 prostate cancer cell line and its taxane resistant variant DU145-DR, in accordance with an embodiment of the disclosure.

FIG. 4A is a photographic image of a clonogenic assay-based dose response for the ligand BT-772 in the PC3 and PC3-DR cell lines, in accordance with an embodiment of the disclosure.

FIG. 4B is a photographic image of a clonogenic assay-based dose response for the ligand BT-772 in the DU145 and DU145-DR cell lines, in accordance with an embodiment of the disclosure.

FIG. 5 is a photographic image of Western blots comparing the ENO1 expression in total protein lysates from taxane sensitive PC3 and DU145 cells and from taxane resistant PC3-DR and DU145-DR cells. The expression of ENO2 is markedly decreased in the taxane resistant cells.

FIG. 6 is a composite of fluorescent images obtained by confocal microscopy showing the localization of ENO1, using a specific anti-ENO1 monoclonal antibody, on the surface of taxane sensitive PC3 and DU145 cells, and on the surface of taxane resistant PC3-DR and DU145-DR cells (green fluorescence). Cell nuclei are counterstained with the DNA binding dye 4′6-diamidino-2-phenylindole (DAPI).

FIG. 7 is schematic representation of a Structure-Activity Relationship (SAR) study, in accordance with an embodiment of the disclosure.

FIG. 8 is a schematic representation of an ENO1 ligand having a lock and key design with radioactive isotopes, in accordance with an embodiment of the disclosure.

### DETAILED DESCRIPTION

The present disclosure provides for several pharmaceutical compositions of ligands of enolase 1 (ENO1) and methods of treating cancer in a subject using these pharmaceutical compositions. So that the manner in which the features and advantages of the embodiments of the compositions and methods disclosed herein, as well as others, which will become apparent, may be understood in more detail, a more particular description of embodiments of compositions and methods is provided. In the following description, numerous details are set forth in order to provide a thorough understanding of the various embodiments. In other instances, well-known processes, devices, and systems may not be described in particular detail in order to not unnecessarily obscure the various embodiments. Additionally, illustrations of the various embodiments may omit certain features or details in order to not obscure the various embodiments.

Boron atoms can be used to facilitate the discovery of new biological activities. With cancer cells known to have high ROS levels, boron-based pharmaceutical compositions can target cancer cells. Boron atoms interact with target proteins producing potent biological activities, and have antioxidant properties by scavenging ROS. As tumor cells have high ROS levels, boron-based drugs can be more specific to tumor cells. Boron-based ENO1 ligands can be modified as linkers for radionuclides targeting ENO1 in the surface of chemosensitive and chemoresistant prostate tumors that lost both PSMA (prostate-specific membrane antigen) and ENO2. Without wishing to be bound to theory, for example, prostate cancer (PCa) that express NEPC markers lose ENO2 expression when they develop taxane resistance.

For example, PCa-DR cells express ENO1, which is recognized by antibodies from European American (EA)-PCa patients but not from African American (AA)-PCa patients. These cells also lose expression of ENO2, which could be preferentially targeted by AA-PCa antibodies. For a subset of AA patients with advanced PCa, their immune system is unable to effectively target ENO1-driven migration and metastasis of chemoresistant PCa cells harboring NEPC markers. However, by expressing high ENO1 levels, these resistant cells can be exploited pharmacologically with boron-based ENO1 ligands leading to attenuated tumor aggressiveness and reduction of PCa burden.

Embodiments of methods of identifying lead ligands for targeting treatment of tumor cell viability in a subject include applying a limited rational design (LRD) approach to generate a small library of only about 10-30 compounds of lead compounds to target ENO1. ENO1 directly regulates key oncogenic pathways involved in cell metabolism, proliferation, AKT signaling, metastasis, tumor immunity, and chemoresistance. Guiding parameters of LRD is based on several factors, including (i) homology modeling of ENO1 crystal structures in silico compounds hypothetically designed and synthesized using known reaction procedures, (ii) new reactions that are developed where existing reaction conditions are not sufficient, and (iii) generation of a small library of compounds without the need to use large existing libraries to evaluate the activity of the compounds. The small chemical library generated from the methods is used to evaluate ENO1 signaling pathways in cancer. Using the LRD approach requires a clear understanding of structure-function relationships and sophisticated modeling tools, minimizing off-target effects caused by random putative “hits.” The methods can include determining, via a computing device, one or more diazaborine-based pharmacophore groups and 2-chloro substituted acetamide derivatives targeting ENO1 based on structure homology modeling. The general structure for the one or more diazaborine-based pharmacophore groups and 2-chloro substituted acetamide derivatives targeting ENO1 is:

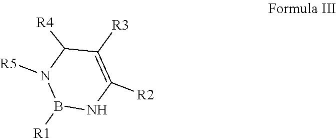

where R1 is an aliphatic moiety that is a straight or branched hydrocarbon moiety up to 5-10 carbon atoms wherein the saturation between any two carbon atoms is a single, double or triple bonds. An aliphatic group preferably contains from 1 to about 8-10 carbon atoms. R2 is an aromatic moiety that is a polycyclic hydrocarbon with conjugated double bonds such as benzene, cyclopentane, or cyclopentadiene. R3 is a heterocyclic moiety, such as furan, pyridine, tetrazole, histidine, benzoxaborole, or diazaborines. R4 is a cyano group (CN). R5 is an acid such as a carboxylic acid or a derivative, such as an amide, an ester, or an aminonitrile.

The structure homology modeling can include Pdbsum, Molsoft, and Schrodinger software, to identify diazaborine-based pharmacophore groups and 2-chloro substituted acetamide derivatives.

A “pharmaceutical composition” refers to a composition of one or more of ligands of ENO1 as an active ingredient, or a pharmaceutically acceptable derivative thereof. In certain embodiments, a pharmaceutical composition contains at least one pharmaceutically acceptable carrier or excipient. The purpose of a pharmaceutical composition is to facilitate the administration of the ligand to a subject. Embodiments include a pharmaceutical composition including a compound of one of the formulae described herein, or a pharmaceutically acceptable derivative thereof, and a pharmaceutically acceptable carrier or excipient. In some embodiments, the pharmaceutical composition includes two or more pharmaceutically acceptable carriers and/or excipients.

In the embodiments, the term “pharmaceutically acceptable derivative” as used herein refers to and includes any pharmaceutically acceptable salt, pro-drug, metabolite, ester, ether, hydrate, polymorph, solvate, complex, and adduct of a compound described herein which, upon administration to a subject, is capable of providing (directly or indirectly) the active ingredient. For example, the term “a pharmaceutically acceptable derivative thereof” of compounds of general formula I, II and III includes all derivatives of the compounds of general formula I, II, and III (such as salts, pro-drugs, metabolites, esters, ethers, hydrates, polymorphs, solvates, complexes, and adducts) which, upon administration to a subject, are capable of providing (directly or indirectly) the compounds of general formula I, II, and III.

In the embodiments, the terms “treatment” or “treating” as used herein includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease. Embodiments of the diseases targeted by the ENO1 ligands include cancer.

The terms “administer,” “administering,” and “administration” refer to introducing a compound, a composition, or an agent (e.g., genetically modified immune cell with a mammalian cell expression vector containing a nucleic acid encoding a mature form of a matrix metallopeptidase or a chemotherapeutic agent) into a subject or subject, such as a human. As used herein, the terms encompass both direct administration, e.g., self-administration or administration to a subject by a medical professional, and indirect administration, such as the act of prescribing a compound, composition, or agent.

An embodiment of a pharmaceutical composition includes a therapeutically effective amount of a ligand of enolase 1. Embodiments of a method of treating cancer in a subject includes administering to the subject the pharmaceutical composition containing a therapeutically effective amount of a ligand of general formula I or a pharmaceutically acceptable derivative thereof. The cancer can be carcinoma, sarcoma, lymphoma, leukemia, or melanoma. The cancer can be of a bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In certain examples, the cancer can be chemosensitive or chemoresistant prostate cancer.

Another embodiment of a pharmaceutical composition includes a therapeutically effective amount of a ligand of enolase 1 having a general formula II or a pharmaceutically acceptable derivative thereof. Another embodiment of a method of treating cancer in a subject includes administering to the subject the pharmaceutical composition containing a therapeutically effective amount of a ligand of general formula II or a pharmaceutically acceptable derivative thereof. The cancer can be carcinoma, sarcoma, lymphoma, leukemia, or melanoma. The cancer can be of a bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In certain examples, the cancer can be chemosensitive or chemoresistant prostate cancer.

Another embodiment of a pharmaceutical composition includes a therapeutically effective amount of a ligand of enolase 1 having a general formula III or a pharmaceutically acceptable derivative thereof, where R1 is an aliphatic moiety, R2 is an aromatic moiety, R3 is a heterocyclic moiety, R4 is a cyano group, and R5 is an acid moiety or a derivative thereof.

Another embodiment of a method of treating cancer in a subject includes administering to the subject the pharmaceutical composition containing a therapeutically effective amount of a ligand of general formula III. The cancer can be carcinoma, sarcoma, lymphoma, leukemia, or melanoma. The cancer can be of a bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In certain examples, the cancer can be chemosensitive or chemoresistant prostate cancer.

In an embodiment, a method of treating cancer in a subject can include administering to the subject a therapeutically effective amount of a pharmaceutical composition having a general formula I. In some embodiments, the cancer treated is prostate cancer (PCa). Other cancer types that express high levels of ENO1 in the cytoplasm and on the surface of cancer cells also can be treated. In these cancers, the presence of ENO1 in the tumors or the presence of antibodies to ENO1 in the blood of the patients carrying these tumors can be indicative of a poor prognosis. These cancers include but are not limited to, bladder cancer, breast cancer, cancer-associated retinopathy, chronic lymphocytic leukemia, colorectal cancer, gastric cancer, glioma, head and neck cancer, liver cancer, lung cancer, multiple myeloma, non-Hodgkin's lymphoma, and pancreatic cancer.

In another embodiment, a method of treating cancer in a subject can include administering to the subject a therapeutically effective amount of a pharmaceutical composition having a general formula II. In some embodiments, the cancer treated is PCa. As described herein, both general formula I and general formula II can further be synthesized into BT-768 and BT-772 compounds. Both BT-768 and BT-772 have anti-cancer effects with low μM cytotoxic activity.

### Making of Pharmaceutical Composition BT-768 and BT-772

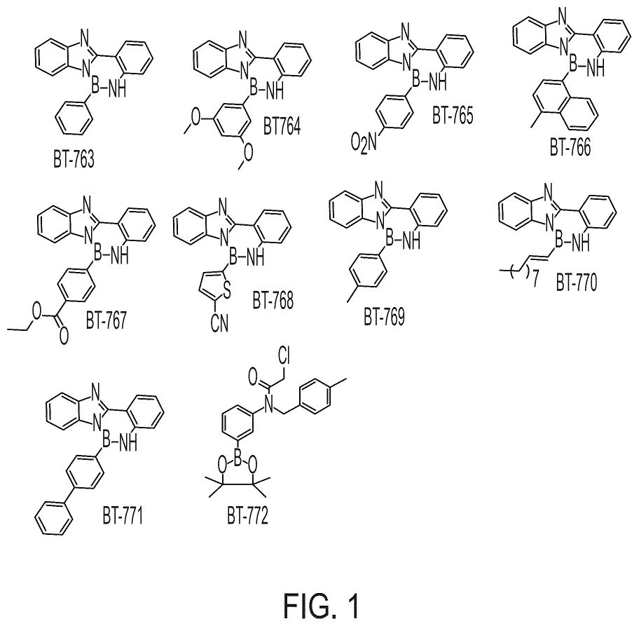

The lead ENO1 ligands, Diazaborine-based pharmacophore group and 2-Chloro substituted acetamide derivatives, further can be synthesized into boron-based ligands. Ten compounds were first synthesized. FIG. 1 shows the structure of these compounds. FIG. 1 is a schematic representation of the general structure of ten compounds synthesized from Diazaborine-based pharmacophore group and 2-Chloro substituted acetamide derivatives. The ten ENO1 ligands generated include: BT-763, BT-764, BT-765, BT-766, BT-767, BT-768, BT-769, BT-770, BT-771, and BT-772. In making these ten compounds, for each reaction step, the new compounds were characterized using proton (1H), carbon (13C), and high-resolution mass spectrometry (HRMS). High-performance liquid chromatography (HPLC) was used to assess the purity of diazaborine-based pharmacophore group and 2-Chloro substituted acetamide derivatives. The biological activity of the compounds was then evaluated.

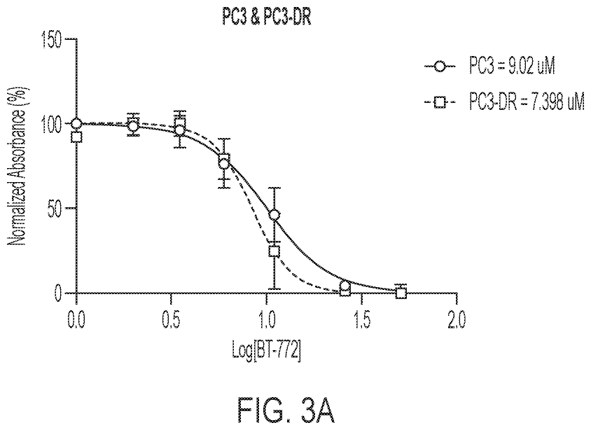

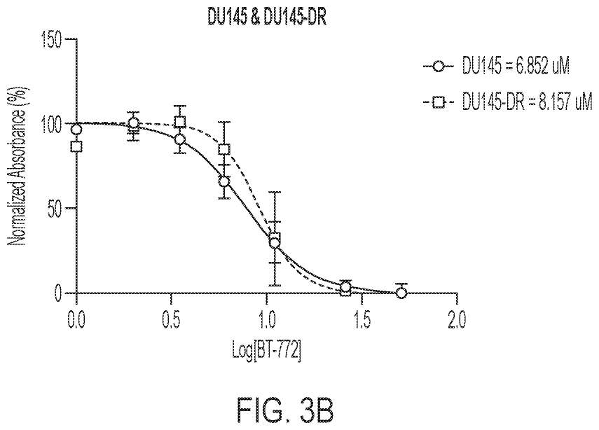

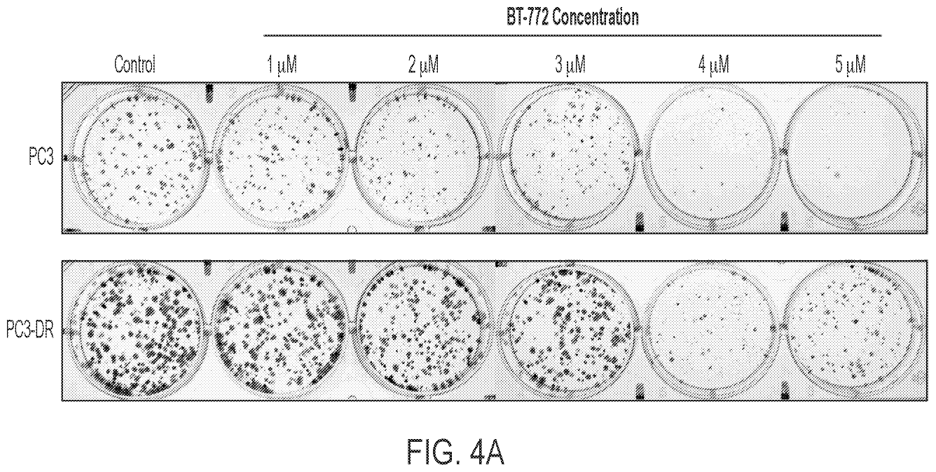

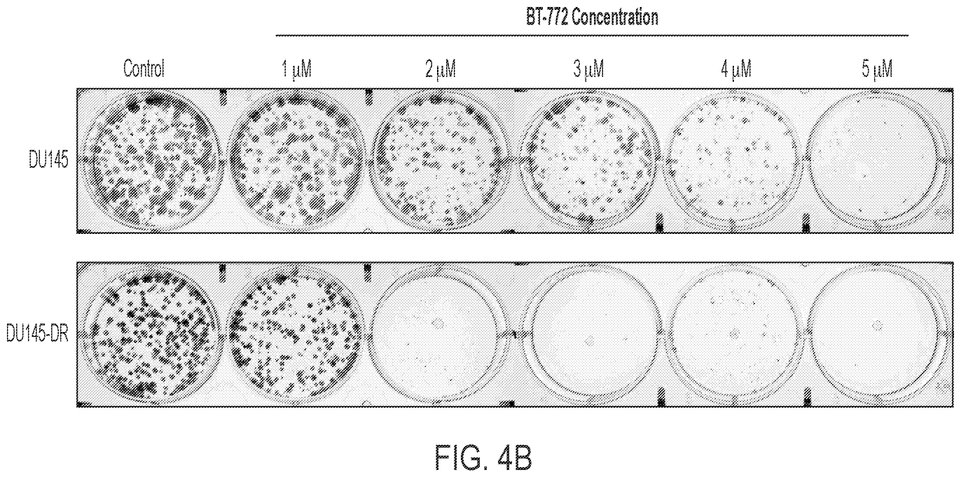

Out of the initial ten compounds, two target-to-hit specific ligands were identified, BT-768 and BT-772, from the diazaborine-based pharmacophore group and 2-Chloro substituted acetamide derivatives. BT-768 and BT-772 were selected for further studies based on their in vitro cytotoxicity and anti-proliferative data in docetaxel resistant PCa cell lines (see FIG. 3A and FIG. 3B, and FIG. 4A and FIG. 4B). As a result, BT-768 and BT-772 were found to have higher cytotoxic than the other eight compounds. The boron-based ligands BT-768 and BT-772 cytotoxic activity in parental and DR cell lines (PC3 and DU145) were then measured. The results suggest that both boron-based ENO1 ligands BT-768 and BT-772 showed a dose-dependent reduction in cell viability.

### Pharmaceutical Composition BT-768

In an embodiment, a pharmaceutical composition contains a therapeutically effective amount of a ligand of a glycolytic enzyme enolase 1. The ligand has a general formula I or a pharmaceutically acceptable derivative thereof. In the embodiment, the pharmaceutical composition includes the compound of general formula I with international union of pure and applied chemistry (IUPAC) name 5-(benzo[e]benzo[4,5]imidazo[1,-c][1,3,2]diazaborinin-6 (5H)-yl)thiopene-2-carbonitrile. The structure is:

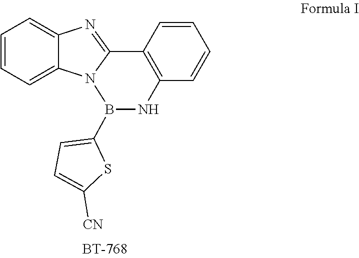

Formula I or a pharmaceutically acceptable derivative thereof is a ENO1 ligand and is a boron-based small molecule inhibitor. Dosing information can be obtained based on results from in vitro anti-cancer studies (assays using cancer cell lines and patient-derived organoids treated with BT-768) and in vivo studies (using cell line-derived and patient-derived tumor mouse xenografts treated with BT-768).

In another embodiment, a method of treating cancer in a subject can include administering to the subject a pharmaceutical composition that has a general formula I. The cancer can be a carcinoma, sarcoma, lymphoma, leukemia, or melanoma. The cancer can be of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. As described herein, the cancer can be PCa.

### Pharmaceutical Composition BT-772

The second of the two pharmaceutical compositions identified, from both Diazaborine-based pharmacophore group and 2-Chloro substituted acetamide derivatives, is BT-772. In another embodiment, a pharmaceutical composition can contain a therapeutically effective amount of a ligand of a glycolytic enzyme enolase 1. The pharmaceutical composition has a general formula II or a pharmaceutically acceptable derivative thereof. In the embodiment, the pharmaceutical composition includes the compound of general formula II with international union of pure and applied chemistry (IUPAC) name 2-chloro-N-(4-methylbenzyl)-N-(3-(4,4,5,5-tetramethyl-1,3,2-dioxaborolan-2-yl)phenyl) acetamide. The structure is:

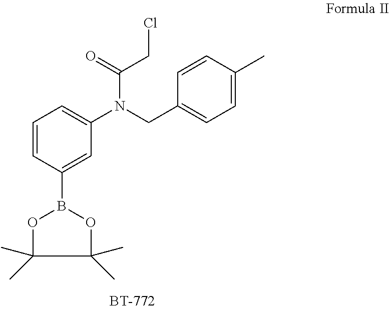

Formula II or a pharmaceutically acceptable derivative thereof is a ENO1 ligand and is a boron-based SMI. Dosing information can be obtained based on results from in vitro studies (assays using cancer cell lines and patient-derived organoids treated with BT-772) and in vivo studies (studies using cell line-derived and patient-derived tumor mouse xenografts treated with BT-772).

In another embodiment, a method of treating a cancer in a subject can include administering to the subject a pharmaceutical composition that has a general formula II. The cancer can be a carcinoma, sarcoma, lymphoma, leukemia, or melanoma. The cancer can be of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. As described herein, the cancer can be PCa.

BT-768 and BT-772 are limited initial compounds identified. Thus, BT-768 and BT-772 can be used as chemical templates to generate new compounds or derivatives with improved anti-cancer effects in the low nanometer (nM) range. For example, compound BT-768 contains 4 rings: A (benimidazole), B (phenyl), C (diazaborine), and D (substituted heterocycles). Additional compounds can be generated by changing each ring system systematically to increase solubility, decrease toxicity, and limit off-target effects. Structure activity relationship (SAR) studies can identify more compounds that have similarly low micrometer (M) cytotoxic activity. Lipinski's rule of five and other chemical tools to develop hit-to-lead molecules is the guiding principle on which the SAR studies are based. Compounds can be designed with considerations for (1) biological activity, (2) ease of synthesis, (3) moderate compound complexity and (4) minimize potential toxic effects. An iterative process can be used to design clinical grade pharmaceutical compositions that are less toxic, have low μM cytotoxic activity with improved PK/PD profiles, and more specific to ENO1 in a go-no-go approach.

BT-768 and BT-772 compounds can be developed through clinical trials to determine the efficacy in treatment of cancer. Development of BT-768 and BT-772 and their analogues include (1) designing and synthesizing of new boron-containing compounds based on the chemical structures of BT-772 and BT-768, (2) evaluating the in vitro anticancer effects of the identified pharmaceutical compositions on cancer cell properties including cell proliferation, clonogenicity, tumorsphere/organoid formation capacity, migration, invasion, metabolism, plasminogen activity, and gene expression, (3) evaluating the in vivo effects of the pharmaceutical compositions on tumor growth properties and overall toxicity using cancer cell line-derived mouse xenograft models, such as for PCa, and patient-derived xenograft models, (4) labeling the pharmaceutical compositions with a radionuclide to study the compositions biodistribution in vivo and the compositions potential to be used as PET/CT theranostic agents in xenograft models of PCa, or (5) translating the lead compounds, such as BT-768 and BT-772, to the clinic through clinical trials to determine the efficacy in cancer treatments, such as advanced PCa treatment, or other cancers modulated by ENO1 signaling pathways.

Using the limited rational design (LRD) approach, small molecules can be developed by homology modeling. Customized computational protein structure modeling programs (e.g., M4T, MMM, and Mutate) and standard programs (e.g., Autodock4, Surflex-Dock, ICM, PESO, SFC, etc.) can facilitate the identification of virtual ENO1 target pharmaceutical compositions. The newly identified compounds then can be synthesized using chemical procedures known to one skilled in the art.

Prostate cancer (PCa) that expresses NEPC markers has lost the expression of PSMA (prostate-specific membrane antigen). As the PSMA expression in the cell's surface has been lost, the target for this life-saving theranostics therapy is no longer present. Then PSMA radioligand therapy is no longer an option for these patients. Therefore, the development of boron-based ENO1 ligands purposefully designed to be linked to radioactive isotopes is novel and transformative. These boron-based ENO1 ligands are designed as theranostics (therapy and diagnostics) agents. After synthesis of the additional boron-based ENO1 ligands, the pharmaceutical compositions can be characterized using nuclear magnetic resonance (NMR), HPLC, HRMS, and elemental analysis for purity and structural configuration.

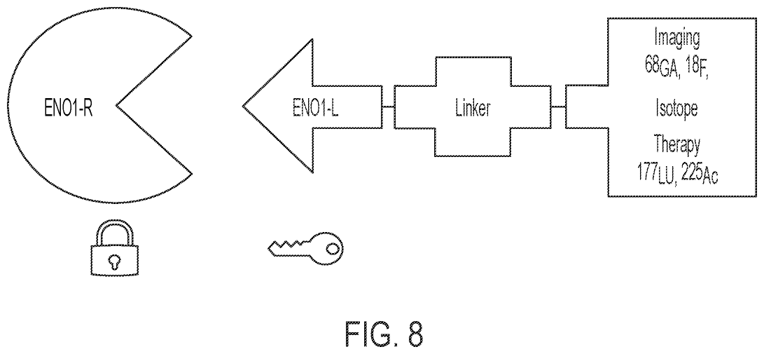

These boron-based ENO1 ligands can be labeled with contrast agents allowing for the use of imaging techniques, such as PET, SPECT, and fluorescence, that enable quantification of tumor-localized boron and their use as theranostic agents. FIG. 8 is a schematic representation of an ENO1 ligand having a lock and key design with radioactive isotopes. The lock and key design allow for the diagnosis and treatment of cancer using the same ENO1 molecule by switching the imaging and therapeutic isotopes. The ability to target the ENO1 receptor on the surface of the chemoresistant prostate cells represents a new lifeline to patients with advanced, chemotherapy-resistant, PSMA-negative neoplastic decease without any other therapeutic options. Of particular importance is that ENO1 is also expressed in a wide variety of cancers, potentially impacting many patients beyond prostate cancer. In certain embodiments, these boron-containing compounds are used as boron-delivery agents for BNCT for cancers. The pharmacologic and chemical behavior of these boron-based ENO1 ligands make them especially advantageous for BNCT.

### EXAMPLES

Various examples are described to illustrate selected aspects of the various embodiments of pharmaceutical composition ENO1 ligands and boron-based SMIs, including pharmaceutical compositions and methods of treating cancer using the pharmaceutical compositions.

### Example 1—Synthesis of Pharmaceutical Compositions (General Formula I, II)

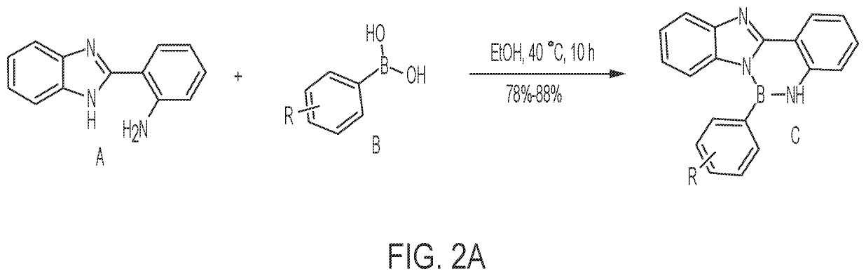

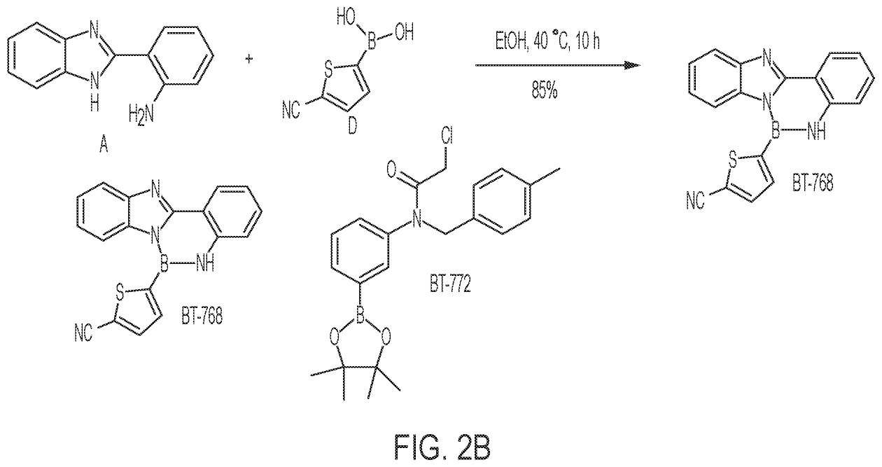

In Example 1, a procedure for the synthesis of diazaborinine is disclosed and described including embodiments of the pharmaceutical composition targeting ENO1 that contain the general formula I. FIG. 2A and FIG. 2B are a schematic representation of the synthesis of diazaborinine. Diazaborinine was synthesized using a clean oven dried 25 milliliter (mL) round-bottom flask charged with 2-(1 H-benzo[d]imidazol-2-yl) aniline A (1.0 equiv.) and phenyl boronic acid derivative B (1.0 equiv.), shown in FIG. 2A. The round-bottom flask charged with A and B was then dissolved in ethanol. Following the dissolvement of A and B in ethanol, the reaction mixture of A and B was then stirred at 40 Celsius (C) for 10 hours. After the reaction was completed, ethanol was first evaporated, then the reaction mass was then cooled to ambient temperature. When the reaction mass reached ambient temperature, the reaction mass was diluted with 30 ml of water. The reaction mass was then extracted three times using 20 ml of dichloromethane to create a combine organic layer. The combined organic layer was then dried with anhydrous Sodium Sulfate (Na₂SO₄) and allowed to evaporate under normal atmospheric pressure. The remaining crude material was purified by column chromatography using an eluent, 100% ethyl acetate to afford desired product C, shown in FIG. 2A. 2-Chloro substituted acetamide derivatives, BT-768, and BT-772 was synthesized using the same procedure as described herein.

### Example 2—MTT Assay-Based Dose-Response Curves for BT-772

FIG. 3A and FIG. 3B, for example, are graphical representations of 3-(4,5-dimethylthiazol-2-yl)-2,5-diphenyl-2H-tetrazolium bromide MTT assay-based dose response curve for the ligand BT-772. FIG. 3A and FIG. 3B represent the average of three independent experiments in triplicates normalized to untreated control assays. As shown in FIG. 3A and FIG. 3B, cell viability was measured by MTT assay. In FIG. 3A, PC3 and PC3-DR cells were treated with increasing concentrations of BT-772 for twenty-four hours. The graphical representation, in FIG. 3A, shows that the IC50s (drug concentration causing 50% inhibition) of the lead molecules (BT-772) have low μM (5-10 μM) cytotoxic activity.

In FIG. 3B, DU145 and DU145-DR cells were treated with increasing concentrations of BT-772 for twenty-four hours. The graphical representation in FIG. 3B shows that the IC50s of the lead molecules (BT-772) have low μM (5-10 μM) cytotoxic activity. Similarly, BT-768 shows a similar dose-dependent reduction in cell viability.

### Example 3—Clonogenic Assay-Dose Response for BT-772

FIG. 4A and FIG. 4B, for example, show images of clonogenic assays, used primarily to determine cancer cell colony formation capacity and proliferation. In FIG. 4A, PC3 and PC3-DR cells were grown at low density in 6-well plates and allowed to form clones for 10 days. Cells were treated with low micromolar concentrations of BT-772 and clonogenicity was noticeably inhibited at concentrations above 3 M in both cell lines. These observations demonstrate the ability of drug-sensitive and drug-resistant PC3 prostate cancer cells to proliferate as clones can be blocked at low micromolar concentrations, thus demonstrating the ability of these compounds to inhibit prostate tumor growth.

In FIG. 4B, DU145 and DU145-DR cells growing as colonies were treated with low micromolar concentrations of BT-772 and clonogenicity was noticeable inhibited at concentrations above 3 μM in DU145 cells and at concentrations above 1 μM in DU145-DR cells. These observations demonstrate the ability of drug-sensitive and drug-resistant DU145 prostate cancer cells to proliferate as clones can be blocked at low micromolar concentrations, thus demonstrating the ability of these compounds to inhibit prostate tumor growth.

### Example 4—Study of Expression of ENO1 and ENO2 in Taxane Sensitive and Taxane Resistant PCa Cells

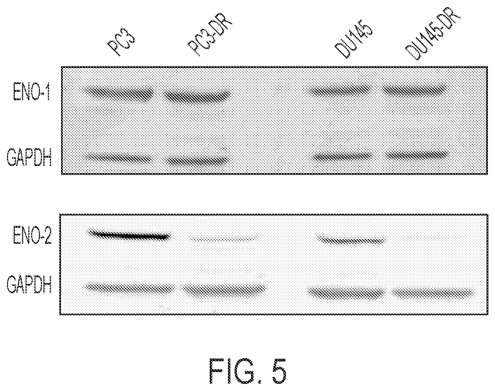

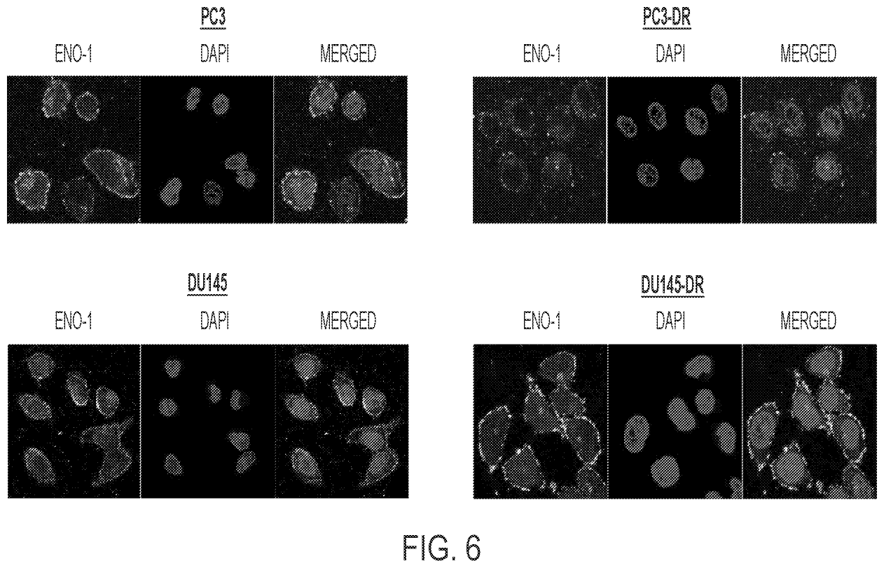

FIG. 5 shows Western blots of protein lysates from taxane sensitive PC3 and DU145 cells and taxane resistant PC3-DR and DU145-DR cells probed with antibodies to ENO1 and ENO2. Proteins were separated by sodium dodecyl sulfate polyacrylamide gel electrophoresis (SDS-PAGE) and then transferred to polyvinyl difluoride (PVDF) membranes which were then probed with commercially available monoclonal antibodies to ENO1 or ENO2. Antibody reactivity against ENO1 or ENO2 was detected after incubating the membranes with a secondary anti-mouse antibody and band detection by chemiluminescence. The blots show robust expression of ENO1 in PC3, PC3-DR, DU145 and DU145-DR cells. The expression of ENO2 was markedly decreased in the taxane resistant cell lines PC3-DR and DU145-DR. This data suggests that development of taxane-based chemotherapy resistance may be associated with downregulation of genes that control ENO2 expression, which may include genes that promote the development of neuroendocrine features. Downregulation of ENO2 expression in docetaxel-resistant PCa cells is not expected to negatively impact the effectiveness of the boron-based compounds as the expression and surface localization of ENO1, the primary target of these compounds, are robust in these cells (FIG. 5 and FIG. 6). Further, the observed efficacy of BT-772 in inducing cytotoxic and anti-proliferative effects in docetaxel-resistant PCa cells (FIG. 3A, FIG. 3B, FIG. 4A and FIG. 4B) indicates that the loss of ENO2 in the chemoresistant cells is not likely to affect the efficacy and choice of these boron-based compounds. The protein glyceraldehyde-3-phosphate dehydrogenase (GAPDH) was used as a loading control for SDA-PAGE. Its expression was constant across the four cell lines in the blots for both ENO1 and ENO2.

### Example 5—Cell Surface Localization of ENO1 in Prostate Cancer Cells

FIG. 6 shows immunofluorescence images obtained by confocal microscopy showing the localization of ENO1 in the surface of taxane sensitive PC3 and DU145 cells and taxane resistant PC3-DR and DU145-DR cells. The immunostaining of ENO1 was achieved by incubating a commercially available monoclonal anti-ENO1 antibody with paraformaldehyde fixed cells growing in glass coverslips followed by incubation with an anti-mouse secondary antibody labeled with fluorescein isothiocyanate (FITC) and mounting of the coverslips in glass slides. The images show that ENO1 is present in the surface of the four cell lines evaluated (green staining), with the strongest fluorescent signal detected in the taxane resistant DU145-DR cells. To visualize the cell nuclei, cells were counterstained with the DNA binding dye DAPI. The surface localization of ENO1 in these prostate cancer cells provide a strong rationale for designing, synthesizing, and characterizing ENO1 ligands that could be used as theranostic agents to treat advanced prostate tumors that express ENO1 in their surface but that lack surface expression of ENO2 and PSMA. These boron-based ENO1 ligands can be labeled with contrast agents allowing for the use of imaging techniques, such as PET, SPECT, and fluorescence, that enable quantification of tumor-localized boron and their use as theranostic agents.

### Example 6—SAR Study of BT-768

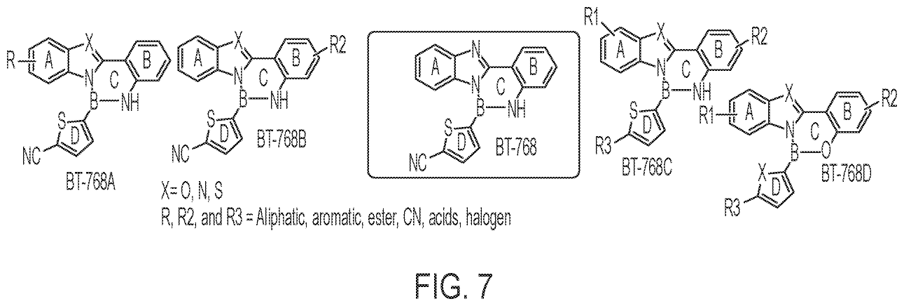

FIG. 7 is a graphical representation of a structure activity relationship (SAR) study and homology model of BT-768. As shown in FIG. 7, SAR studies and homology modeling can identify more pharmaceutical compositions that have similarly low μM cytotoxic activity. The compound has an X group that includes O, N, and S. The compound also has R1, R2, and R3 groups that include one or more of an aliphatic, an aromatic, an ester, a CN, an acid, or a halogen moiety. The halogen moiety can be one of fluorine, chlorine, bromine, or iodine.

### Example 7—Determine Mechanisms Underlying the Differential Reactivity and Antitumor Effects of Anti-ENO Autoantibodies in AA and EA Men with PCA

Circulating antibodies to ENO, present in cancer patients, is associated with improved or poor patient outcomes depending on the cancer type and can have anti-tumor functions. Serological proteomic analysis (SERPA) is used for profiling circulating anti-TAA autoantibodies in racially diverse PCa patients. This approach involves probing patient sera against PCa cell proteins using 2-dimensional Western blots (2D-WB) followed by MS/MS identification of immunoreactive protein spots. The immunoreactivity of randomly selected sera from AA-PCa patients (n=28) and EA-PCa patients (n=27) is compared against PC3-PCa cellular proteins by WB under identical conditions. AA patient sera show stronger immunoreactivity and antibody frequency against ENO (identified by SERPA) than EA patient sera (29% versus 3.7%). The reactivity of the AA-PCa sera against ENO in PC3 cell lysates resembles that of an anti-ENO1 mAb antibody and is abolished by ENO1 knockdown. BT-768 and BT-772 and their derivatives can be used in these studies to determine whether their binding to ENO1 alters the immunoreactivity of anti-ENO1 human and commercial mAb antibodies.

A larger cohort (194 AA-PCa sera, 135 EA-PCa sera, and 183 non-PCa matched controls) is then evaluated by ELISA against human recombinant ENO1 (Rec-ENO1). The results suggest that the frequency of ENO1 antibodies is higher in the PCa group compared to the non PCa group. However, this frequency is higher in the EA-PCa group than in the AA-PCa group, which is inconsistent with the WB results using PC3 lysates. WB representative AA-PCa and EA-PCa sera are then probed against Rec-ENO1 and PC3 ENO. The results suggest that AA-PCa sera strongly react with cellular ENO but poorly with Rec-ENO1. By contrast, EA-PCa sera reacts strongly with Rec-ENO1 and moderately with cellular ENO. BT-768 and BT-772 and their derivatives can be used in these studies to determine whether their binding to ENO1 interferes with the immunoreactivity of anti-ENO1 human antibodies with Rec-ENO1 or cellular ENO.

Next, the reactivity of representative AA-PCa and EA-PCa ENO-positive sera is analyzed against a panel of PCa cell lines using WB. The results suggest that mAB-ENO1 and EA-PCa sera react with ENO1 across the cell line panel. However, AA-PCa sera show differential reactivity across the panel, with strong ENO immunoreactivity in the AA cell line MDA-PC-2b and the EA cell lines PC3 and DU145. Unlike the EA-PCa sera, AA-PCa sera does not react against ENO in docetaxel (DTX) resistant PC3-DR and DU145-DR cell lines. DR and DU145-DR cell lines expression of chromogranin A and ENO2 is examined by WB using monoclonal antibodies since PC3-DR and DU145-DR cell lines are known to express NEPC markers. 22RV1 & 22RV1-DR cell line pair is also analyzed. The results suggest that while both the DTX-sensitive and DTX-resistant (DR) cell lines retained chromogranin A, all the DR cell lines lose ENO2 reactivity. This pattern is identical to that produced by the AA-PCa ENO antibodies, raising the interesting possibility that they may preferentially target ENO2, with EA-PCa antibodies preferentially targeting ENO1. Alternatively, it is possible that this differential immunoreactivity is due to differences in recognition of ENO1 PTMs by AA-PCa and EA-PCa sera. MS/MS differences in ENO1 PTMs between PC3 and PC3-DR cells are detected with PTMs unique to either parental or DR cells. These results suggest that the MS/MS analysis of immunoreactive spots in 2D WB and the ENO knockdown is not distinguishable between ENO1 and ENO2 given their sequence and SDS PAGE migration similarities, a suggestion that these two isoforms is possibly differentially recognized by AA-PCa and EA-PCa sera and the reactivity of AA-PCa anti-ENO1 antibodies, but not that of EA-PCa antibodies is possibly dependent on PTMs present in cellular ENO1 but not in Rec-ENO1.

To determine whether AA-PCa and EA-PCa anti-ENO antibodies have differential effects on the migration of PC3-DR and DU145-DR cells, IgG antibodies from 6 EA-PCa sera and 7 AA-PCa sera are purified and pre-absorbed with Rec-ENO1 to neutralize their reactivity, since ENO1 localizes to the tumor cell surface to promote migration and metastasis. EA-PCa anti-ENO sera and their IgGs prevent PC3-DR cell migration, whereas AA-PCa anti-ENO sera and IgG fail to prevent it. Pre-absorption of EA-PCa sera and IgG with Rec-ENO1 abolish their cell migration inhibitory effects. This is confirmed with mAB ENO1 and in DU145-DR cells which suggests that EA PCa ENO antibodies target surface ENO1 in DR cells, whereas AA-PCa antibodies may target ENO2, whose expression is lost from these cells. BT-768 and BT-772 and their derivatives can be used in these studies to compare their anticancer properties, such as clonogenicity, migration, and invasion, with those of anti-ENO1 human and commercial mAb antibodies.

The differences in antibody reactivity between AA and EA patients indicate either differential recognition of distinct isoforms of ENO (ENO1 vs ENO2) or post-translationally modified (PTM) forms of ENO1.

### Example 8—Immune Profiling of PCa Sera

A large cohort of PCa patients is screened and matched with controls for the presence of anti-ENO1 or anti-ENO2 serum antibodies. Over 800 serum samples from AA men and over 600 samples from EA men (>21 years old, with and without PCa) are accrued from clinics, biorepositories, and community health fairs. Prostate specific antigen (PSA) levels are determined for each sample to rule out possible PCa in the control non-PCa group. DNA is available for most samples if ancestry analysis is needed. Clinical and sociodemographic data are also available, which allows correlating antibody presence with specific parameters. Detection of serum ENO antibodies is done by ELISA using purified recombinant ENO1 and ENO2 proteins as substrates (commercially available).

Immunoreactive sera are then confirmed by WB against recombinant ENO1 and ENO2 and by SERPA. Selected confirmed anti-ENO1 or -ENO2 sera (n=10 each) are next probed by WB against lysates from a diverse PCa cell line panel that includes 3 EA DR cell lines. Two AA DR cell lines (RC-77T/E-DR and MDA-PCa-2b-DR) can be developed as disclosed herein. In addition, lysates from prostate tumor organoids developed from patient-derived xenografts (PDXs) are used. For example, A-PCa and 2 EA-PCa PDXs (1 DTX-sensitive and 1 DTX-treated) for each race are provided by Champions Oncology. Patient immunoreactivity against ENO1 and ENO2 is then compared with the reactivity of commercial anti-ENO1 or ENO2 mABs in individual cell lines or organoids. To confirm that AA and EA patient sera differentially recognize ENO1 or ENO2, CRISPR knockouts are completed in selected cell lines using kits specific for ENO1 (Santa Cruz Biotechnology) and ENO2 (Origene), then probe selected AA and EA patient sera and ENO1 or ENO2 mABs against the lysates by WB. It is anticipated that PCa cell lines lacking ENO1 are resistant to treatment with BT-768 and BT-772 and their derivatives.

### Example 9—Cell Surface Localization of ENO1 in Prostate Cancer Cells Detected with Human Autoantibodies

The cell migration inhibitory effects of anti-ENO1 antibodies from EA-PCa patients suggests immune targeting of surface ENO1 in the DR cells. Purified IgG antibodies from patient sera (specificity confirmed by IgG pre-absorption with Rec-ENO1) is used to determine ENO1 surface localization in selected parental and DR cell lines via flow cytometry and confocal microscopy, following standard procedures in a lab, as demonstrated with commercial monoclonal antibodies in Example 5 and FIG. 6. Further confirmation of ENO1 cell surface localization is achieved by immunoblotting reactivity of patient antibodies and mAB-ENO1 against the membrane, cytoplasmic, and nuclear fractions from selected cell lines and patient-derived organoids (Cell Fractionation Kit, Cell Signaling). Controls for individual fractions are then identified from the extensive antibody collection in the lab. BT-768 and BT-772 and their derivatives can be used in these studies to determine their effect on ENO1 cancer cell surface localization.

### Example 10—Effects of Anti-ENO Antibodies on PCa Cell Aggressive Properties

Anti-ENO IgGs from selected EA-PCa and AA-PCa sera, and from commercial sources, are evaluated for their inhibitory properties on the proliferation, migration, invasion, clonogenicity, and tumorsphere formation properties of selected parental and DR cell lines, as described in Examples 2 and 3 and shown in FIGS. 3A, 3B, 4A, 4B. Anti-ENO IgGs effects on patient-derived organoid growth is also evaluated. To identify gene pathways linked to these effects, RNAseq profiling and pathway analysis on selected cell lines can be completed using standard procedures optimized in the labs of the inventors. These effects can be compared with those produced by BT-768 and BT-772 derivatives in similar studies.

### Example 11—ENO1/ENO2 Expression in PCa Tissue Microarrays (TMAs)

To assess differences in ENO1 and ENO2 expression in PCa and normal prostate tissues a multiplex immunohistochemistry (IHC) is completed using fluorescently labeled specific antibodies in race related PCa TMAs containing 76 cores (19 AA tumors, 19 EA tumors, and their respective normal tissues). If significant differences are detected after pathology scoring, a larger cohort using TMAs from the Prostate Cancer Biorepository Network or other repositories can be used.

### Example 12—Anti-ENO1 Enzymatic Activity Assays

Metabolomics is used to quantify new SMIs metabolic perturbation. A procedure to understand the metabolic consequences of inhibiting glycolysis via enolase inhibition is performed using untargeted metabolite profiling on ENO1-KO and ENO1-WT cells treated with BT-768 and BT-772 derivatives. This approach permits real-time evaluation of ENO1 inhibition. The procedure suggests that the accumulation of metabolites, such as 2-phosphoglycerate after anti-ENO1 treatment is an indication of ENO1 inhibition.

Colorimetric assay kits is used to assess what isoform is metabolically active by measuring ENO1 activity in total cell lysate from ENO1-suppressed and ENO1-WT cells treated with BT-768 and BT-772 derivatives. ENO1 activity is determined via a coupled reaction to the consumption of NADH in an assay buffer.

### Example 13—Effects of ENO1 SMI on PCa Cell Aggressive Properties

### Tumorsphere Formation Assay

BT-768 and BT-772 compounds capable of limiting tumor growth are analyzed using selected PCa cell lines, and primary patient derived PCa tumorspheres/organoids, which are considered a translational system. Tumorsphere formation and quantitative analysis is determined after anti-ENO1 treatment with increasing concentrations of BT-768 and BT-772 derivatives.

### Cytotoxicity and Proliferation Assays

Cell lines and patient derived tumorspheres/organoids are used to determine changes in cell proliferation and viability after anti-ENO1 treatment with increasing concentrations of BT-768 and BT-772 derivatives. After anti-ENO1 treatment, residual cell viability is determined by trypan blue dye exclusion test. Cytotoxicity is assessed by MTT assay or similar cytotoxicity assays. IC50 values are derived from the generated viability curves. The effects of anti-ENO1 treatments on cell proliferation can be assessed by clonogenic assays.

### SMI Impact on PCa Cellular Functions

BT-768 and BT-772 compounds are evaluated for their ability to induce cell death (apoptosis assays), and inhibit cell cycle progression, clonogenicity, invasion, and migration.

### Alternative Testing

The specificity of ligands for ENO1 (or even ENO2) is further established using cell binding assays (Cellular Thermal Shift [CTS] and Drug Affinity Responsive Target Stability [DARTS]), Isothermal Titration calorimetry (Rec-ENO1 or Rec-ENO2+SMIs) and other biophysical assays.

All assays are done in at least 3 independent experiments, each with a least 3 biological replicates, and appropriate controls. RNA-seq for each individual experimental sample is conducted in three independent biological replicates, and genes with statistically significant (t-test) fold change >2 in expression are considered differentially regulated and independently validated. WB is quantified by densitometry in at least three independent experiments.

Percent change values between treatment groups are analyzed by unpaired Student's t-test and non-parametric Mann-Whitney-U test. SPSS Statistics Software V22.0 and GraphPad Prism 6 is used for all statistical analyses, and P<0.05 is accepted as statistically significant. Comparison of fold changes of protein expression in WB will be done using Student's t-test. Differences in protein expression between tumors and normal tissues is analyzed by Kruskal-Wallis's test. Associations between serum antibody levels or protein tissue expression levels and clinicopathologic parameters is determined by Kendall's tau correlation analysis. Group comparisons in ELISA use ANOVA with Bonferroni correction.

A power analysis of covariance with the power of 80%, level of significance=0.05, and assuming proportion difference of 0.14 for four groups (EA vs. AA, PCa vs. non-PCa) with an estimated sample size of 224 per group is used to estimate the minimum number of human serum samples needed for the ELISA studies.

When ranges are disclosed herein, ranges from any lower limit may be combined with any upper limit to recite a range not explicitly recited, as well as ranges from any lower limit may be combined with any other lower limit to recite a range not explicitly recited, in the same way, ranges from any upper limit may be combined with any other upper limit to recite a range not explicitly recited. Additionally, reference to values stated in ranges includes every value within that range, even though not explicitly recited. Thus, every point or individual value may serve as its own lower or upper limit combined with any other point or individual value or any other lower or upper limit to recite a range not explicitly recited. Other objects, features, and advantages of the disclosure will become apparent from the foregoing drawings, detailed description, and examples. These drawings, detailed description, and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific

What is claimed is:

1. A pharmaceutical composition comprising a therapeutically effective amount of a ligand of enolase 1 having a general formula I or a pharmaceutically acceptable derivative thereof:

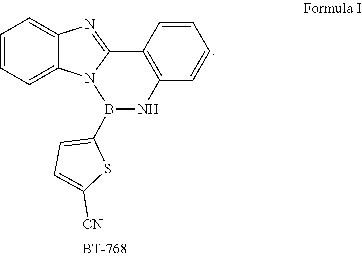

2. A method of treating cancer in a subject, the method comprising administering to the subject the pharmaceutical composition of claim 1.

3. The method of claim 2, wherein the cancer is a carcinoma, sarcoma, lymphoma, leukemia, or melanoma.

4. The method of claim 2, wherein the cancer is of a bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

5. The method of claim 4, wherein the cancer is prostate cancer.

6. A pharmaceutical composition comprising a therapeutically effective amount of a ligand of a glycolytic enzyme enolase 1 having a general formula II or a pharmaceutically acceptable derivative thereof:

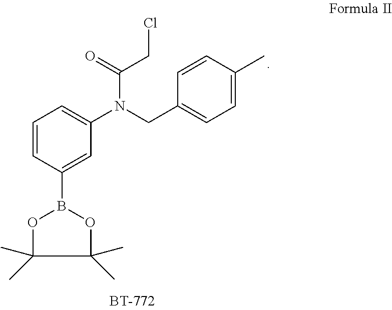

7. A method of treating cancer in a subject, the method comprising administering to the subject the pharmaceutical composition of claim 6.

8. The method of claim 7, wherein the cancer is a carcinoma, sarcoma, lymphoma, leukemia, or melanoma.

9. The method of claim 7, wherein the cancer is of a bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

10. The method of claim 9, wherein the cancer is prostate cancer.

11. A pharmaceutical composition comprising a therapeutically effective amount of a ligand of enolase 1 having a general formula III or a pharmaceutically acceptable derivative thereof: 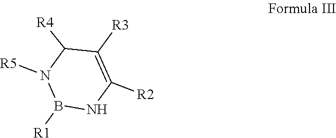
where R1 is an aliphatic moiety, R2 is an aromatic moiety, R3 is a heterocyclic moiety, R4 is a cyano group, and R5 is an acid moiety or a derivative thereof.

12. A method of treating cancer in a subject, the method comprising administering to the subject the pharmaceutical composition of claim 11.

13. The method of claim 12, wherein the cancer is a carcinoma, sarcoma, lymphoma, leukemia, or melanoma.

14. The method of claim 12, wherein the cancer is of a bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

15. The method of claim 14, wherein the cancer is prostate cancer.
